(19) United States
(12) Patent Application Publication
(10) Pub. No.: US 2012/0184974 A1
(43) Pub. Date: Jul. 19, 2012

(54) Supporting and Forming Transitional Material for Use in Supporting Prosthesis Devices, Implants and to Provide Structure in a Human Body

(76) Inventor: Hilton Becker, Boca Raton, FL (US)

(21) Appl. No.: 13/353,960

(22) Filed: Jan. 19, 2012

Related U.S. Application Data
(63) Continuation-in-part of application No. 12/832,161, filed on Jul. 8, 2010.
(60) Provisional application No. 61/351,062, filed on Jun. 3, 2010.

(51) Int. Cl. A61F 2/00

(52) U.S. Cl. 606/151

(57) ABSTRACT

A biocompatible fabric comprised of a strong absorbable material and ultra fine permanent fibers which serve as a collagen support system for prosthesis devices and implants. The permanent fibers stimulate the growth of collagen but are too weak to offer the initial support desired. As the absorbable material dissolves, the collagen formed over the permanent fiber assumes the support role from the absorbable material. The supporting system is natural in appearance and motion without encapsulation or rigidity. The absorbable material can be overlaid with the non-absorbable fiber which after absorption leaves the non-absorbable mesh in an array. The fibers can be discontinuous, loosely woven or embedded in an absorbable material and can be patterned to provide various strengths and degrees of motion and movement. The materials can be coated or infused with materials to reduce infection, provide tissue growth, reduce scar tissue or other medicinal purposes.

### PRIORITY CLAIM

This application is a continuation-in-part of U.S. patent application Ser. No. 12/832,161, filed on Jul. 8, 2010, which claims priority to U.S. Provisional Application No. 61/351,062, filed on Jun. 3, 2010, the disclosure and contents of which are expressly incorporated herein by reference.

### FIELD OF THE INVENTION

This invention relates to a forming and supporting material usable for breast reconstruction following a mastectomy, breast augmentation or modification, or the treatment of breast implant complications, especially capsular contraction and more particularly to a method for forming and supporting a breast implant in a human body. The material may also be utilized in other areas of reconstructive surgery where initial non-yielding, strong support is required or desired to be provided which after a suitable period of time, when healing has occurred, transforms into a flexible matrix material having the characteristic of human tissue acting as a scaffold that allows host tissue in-growth without restriction of elasticity providing flexibility and natural motion.

Implants and methods for breast reconstruction and augmentation are well known and have been used for over forty years. The two primary difficulties with implants have been the issue of supporting and restraining the implant to maintain its location and to supply an extra layer of tissue over the implant while allowing appropriate movement and preventing fibrous scar tissue encapsulation. The problems of providing appropriate support while maintaining the desired movement have been dealt with in many of the patent applications discussed herein. The greater support provided the less natural movement is achieved. This problem is amplified by the creation of scar tissue. When a foreign body is implanted into the body, the implant material is walled off by the response of the human tissue. This is commonly referred to as encapsulation. As the capsule that is formed is scar tissue, it is fairly rigid and in certain cases may actually contract, resulting in hardness around the implant. The encapsulation can also lead to the problem of spherical scar contracture. The scar tissue surrounds the prosthetic device and as it contracts causes the configuration of the implant to be altered as well as creating hardness, discomfort, displacement and pain. Implants are traditionally placed under the muscle or partially under the muscle when performing breast reconstruction. This is done to provide an extra layer of tissue over the implant. By providing a material which acts as a scaffold over the implant it would not be necessary to perform additional surgery to elevate the muscle over the implant.

Numerous approaches have been proposed to address these problems. Cronin, U.S. Pat. No. 3,293,663 filed on Dec. 27, 1966, was one of the first implant patents for breast prosthesis that proposed support use of corrugated fabric against the chest wall, which is anchored to the back portion of the implant. The Perras U.S. Pat. No. 3,665,520 filed on May 30, 1972, later proposed support using a Dacron strip affixed to the back wall of the implant. Frank, U.S. Patent Application No. U.S. 2007/0088434A1 filed on Nov. 29, 2006, proposed support using a sheet of prosthetic material configured to form a sling-shaped receiving area to support the breast implant.

Popov PCT Application WO2007/004214A2 filed Jun. 28, 2006, uses a basket-shaped structure to provide support to an implant or the mammary gland itself.

These proposals provided support but did not fully allow natural motion, nor did they deal with the problem of encapsulation.

A second approach has been to have the implant surface textured or modified to be bio-compatible to provide support and hopefully reduce the problem of encapsulation. This is seen in McGhan, U.S. Pat. No. 6,913,626B2 filed on Jul. 5, 2005, and Agerup, Patent Application. No. 2008/0312739A1 filed on Dec. 18, 2008. There has been only moderate success with such procedures and later studies have indicated it has not achieved the desired objectives.

Another approach has been use of an outer container or pouch to hold the implant prosthesis and interact with the body by in-growth to provide support and try to reduce the occurrence of capsular contracture's inhibiting properties.

The use of sheet or film type materials which are biologically absorbable into the body to provide support has been tried and is disclosed in Naficy, U.S. Pat. No. 4,298,998 filed on Nov. 10, 1981. The use of non-bio-absorbable mesh coated with a bio-absorbable material is seen in Buevich, U.S. Patent App. 2008/0132922A1 filed on Jun. 6, 2008, and using biocompatible but non-bio-absorbable meshes to provide supporting structure is disclosed in Chen, U.S. Patent App. 2009/0082864A1 filed on Mar. 26, 2009, O'Keefe, U.S. Pat. No. 4,936,858 filed on Jun. 26, 1990, and Maxwell, U.S. Patent App. 2009/0125107A1 filed on May 14, 2009. However, the use of these various meshes has not resolved the problem related to breast or other type implants as corrective operation rates to address need for more natural movement and capsular contracture have been unacceptably high.

The use of fabric type materials has been known for some time, the purpose of which has been to provide the function of a permanent re-enforcing structure. These materials have usually incorporated a permanent non-absorbable structure which at times is laced together or coated with various bio-absorbable portions used as filler type material. See Landi, U.S. Pat. No. 5,326,355 filed on Jul. 5, 1994, and Ledergerber, U.S. Pat. No. 4,955,907 filed on Sep. 11, 1990. Typically these fabric type materials contain a weave or structure, usually a knotted mesh, that will be of a biocompatible, non-absorbable material which may have a bio-absorbable component. After absorbance of any bio-absorbable components, the remaining structure which is intended to provide needed support provides insufficient strength and in many cases cause scar tissue to form about the permanent, non-absorbable material resulting in the scar tissue formation, discomfort and hardness, as well as need for corrective surgery. The use of mesh fabrics to provide additional support and use of textured surfaces to promote tissue growth and affix the implant is taught in co-pending U.S. patent application Ser. No. 12/169,000 filed on Jul. 8, 2008 which is a continuation in part of U.S. patent application Ser. No. 12/026,032 filed on Feb. 5, 2008. Also incorporated by reference are co-pending applications U.S. patent application Ser. No. 12/552,352 filed on Sep. 2, 2009, U.S. patent application Ser. No. 12/556,050 filed on Sep. 9, 2009, and U.S. patent application Ser. No. 12/026,032 filed on Feb. 5, 2008. However, none of these prior art attempts have solved the problem of providing both adequate support, strength, elasticity and scaffold function with acceptable control or elimination of capsular contracture, scar tissue formation, discomfort and hardness.

The prior art did not achieve support with flexibility and motion. Clinical surgical re-cutting often had to be performed on previously utilized fabric support to achieve flexibility and motion. This invention eliminates the formation of the typical encapsulation without need for surgical intrusion, and unexpectedly promotes collagen growth to provide support.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, comprises a segmented fabric comprising sections which are permanent fibers which are ultra fine and non-absorbable and sections which are absorbable, such that the fabric transitions from a continuous sheet to an array of non-absorbable fibers which are preferably not connected to each other but may be fused or noted together. As the absorbable portion dissipates, the implant and tissue are provided structural support by the collagen structure that is formed over the non-absorbable fibers. The collagen and the areas between the non-absorbable segments are natural body tissues that can move naturally and elastically. Since the absorbable fabric areas dissipate and the ultra fine fibers do not cause scar tissue growth capsular contracture does not become a problem. The fabric can be conformed into a variety of configurations and reduced to single strands.

It is the desire of this invention by virtue of its unique and novel approach to provide the following benefits to breast implant and other restorative procedures; provide support but allow natural movement, prevent or reduce to nominal levels capsular contracture, allow use of smooth surfaced gel implant prosthesis more effectively, with placement above the muscle, stop or minimize rippling from occurring, provide treatment to encourage in-growth and against potential infections, and provide the scaffold for the forming of a new tissue layer that is permanent but elastic, by simulating natural human supportive tissue.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### DESCRIPTION OF DRAWINGS

FIG. 1 illustrates a transitional fabric material using a hexagonal non-absorbing area separated by an absorbing area.

FIG. 1(a) illustrates the transitional fabric before utilization.

FIG. 1(b) illustrates the transitional fabric after utilization and absorption has occurred.

FIG. 2 illustrates a transitional fabric where the absorbable mesh portion is overlaid on the sections of a non-absorbable material.

FIG. 3 illustrates the fabric utilized for an implant device, shaped in conical fashion.

FIG. 4 illustrates the fabric utilized for an implant device, shaped in a basket or pocket configuration.

FIG. 5 illustrates a mesh utilizing knotted connections representing non-yielding mesh.

FIG. 6 illustrates a mesh utilizing an unknotted weave and intentional breaks in the weave representing a yielding fabric mesh.

FIG. 7(a) illustrates a loose absorbable weave mesh housing ultra fine non-absorbable fiber.

FIG. 7(b) illustrates the mesh of FIG. 7(a) after absorption has occurred with remaining woven non-absorbable ultra fine fiber mesh.

FIG. 7(c) illustrates a thread with absorbable material housing strands of non-absorbable ultra fine fiber.

FIG. 7(d) illustrates the fiber weave of FIG. 7(c) after absorption has occurred and shows the intentional break in non-absorbable ultra fine fibers.

FIG. 8(a) illustrates the non-absorbable ultra fine fibers wrapped around the absorbable material.

FIG. 8(b) illustrates the non-absorbable ultra fine fibers after absorption of the absorbable material has occurred.

FIG. 9 illustrates a sponge-type sheet of absorbable material with non-absorbable ultra fine fibers contained therein.

FIG. 10(a) illustrates the transitional mesh material using a hexagonal absorbing material fully overlaid or interwoven with a non-absorbing ultra fine fibers utilized over an entire area.

FIG. 10(b) illustrates the ultra fine fibers after utilization and absorption has occurred.

FIG. 11(a) illustrates the transitional fabric material using a hexagonal absorbing material overlaid or interwoven partially with a non-absorbing ultra fine fibers utilized over a partial area.

FIG. 11(b) illustrates the ultra fine fibers after utilization and absorption has occurred.

### DESCRIPTION OF THE PREFERRED EMBODIMENTS OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

Collagen is a natural biological material secreted by fibroblasts in the body. Fibroblasts are stimulated to secrete collagen in response to injury, i.e. if the skin is cut, to function as a “cement” to bridge the gap between the cut edges and provide support to the skin. Collagen however is a transitory material and only remains in place as long as the functional tension which it serves to address continues. Thus the initial outpouring of collagen in case of injury or other causing event will stop once the cause is removed and if there is no continuation of the load or tension being placed upon the collagen it will reabsorb into the body. Fibroblasts are stimulated to secrete collagen in response to a piezo-electric current (a micro current found on the surface of any material and which is increased by tension placed on the material). See Blecker, H., Diegelmann, R. F.; The Influence of Tension on Intrinsic Tendon Fibroplasia. Orthopedic Review, 13, 65-71, 1984; Tendon Cell Proliferation in Tissue Culture, J. Hand Surgery; 6:616-619, 1981.

A synthetic fabric mesh comprised of synthetic non-absorbable ultra fine fibers will stimulate fibroblasts in formation and growth of collagen formations when introduced into living tissue. The piezoelectric current on the surface of the fibers cause the needed stimulation for this process. An absorbable fiber will stimulate collagen formulation by this mechanism, however when the fiber is absorbed the stimulus for the collagen formation is no longer present and the collagen originally produced in response to the fiber will be reabsorbed into the body. A non-absorbable fiber will maintain the presence of collagen, with thicker the fiber the more collagen being formed, due to the greater piezoelectric current on the larger surface. However, thicker fibers have numerous problems in human tissue such as formation of scar tissue and rigidity, palpability, irritation of tissue with extrusion and infection.

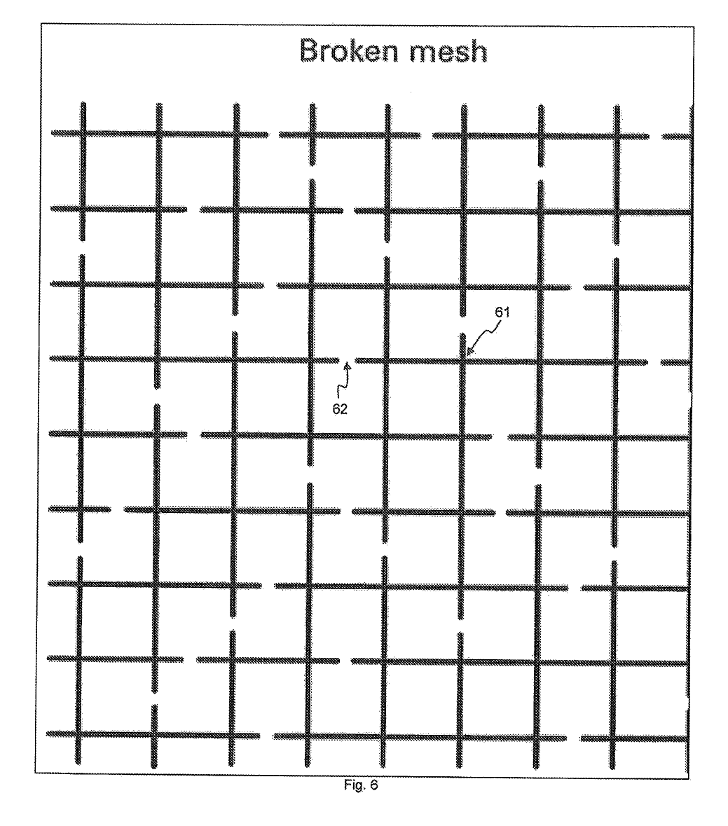

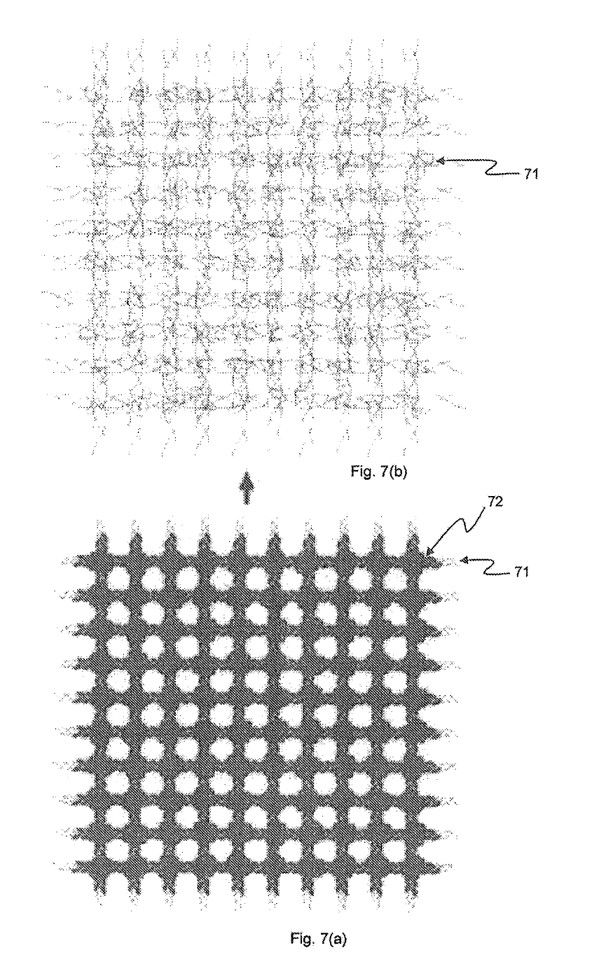

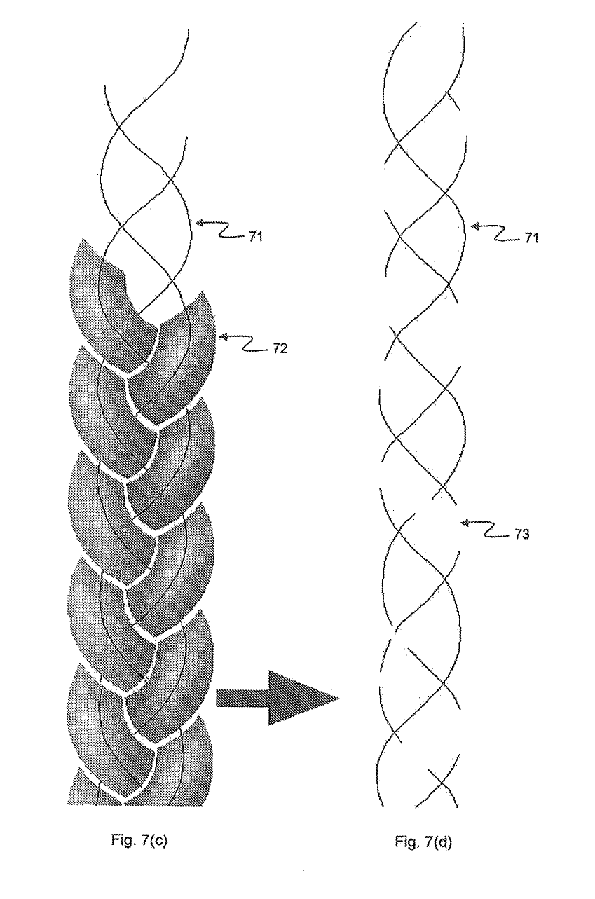

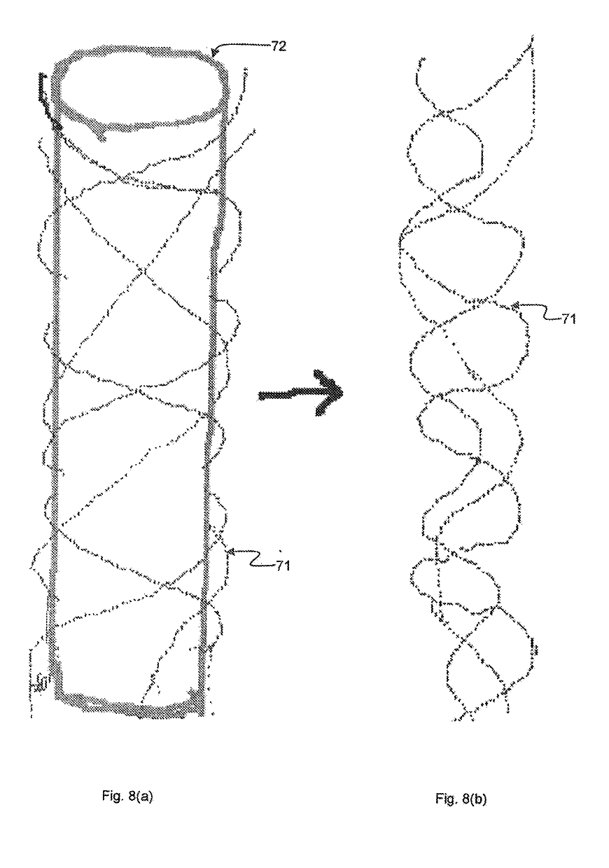

The present invention overcomes the disadvantages of the prior art by incorporating a ultra fine non-absorbable synthetic fiber in conjunction with an absorbable supportive material that provides the needed initial support when a breast implant is being inserted or reconstructive surgery is being performed. Surprisingly, the ultra fine synthetic fiber stimulates the formation of collagen which then creates a scaffold for collagen to form that will provide the needed support. Initially, the ultra fine fiber by itself is too weak to function as a support material and the function of support is taken up by the absorbable material which has the needed strength. The ultra fine fibers however stimulate the collagen formation. As the absorbable material dissipates over several months the load or tension bearing transfers to the collagen which is stimulated in growth by the application of the tension load. Thus a natural body structure is formed to take up the load being transferred from the absorbable material. Since the ultra fine fiber is so small scar tissue formation and capsule constriction does not occur. Thus we are left after several months with a support structure of collagen which can provide the support needed and is elastic allowing natural movement. In the following figures we see the fibers, meshes and fabrics used for combining non-absorbable and absorbable material to form various fabric configurations. The preferred embodiments for the ultra fine fiber as found in FIGS. 7(c), 7(d), 8(a), 8(b) 9, 10(a), 10(b), 11(a) and 11(b), make use of the ultra fine fiber in broken and random arrays but it can be used in any of the figures shown or variations of the shown applications where the absorbable material is strong enough to meet the strength requirements. The finer the size of the fiber the better the result that can be obtained. The preferred range for the fiber is from about 0.01 of a millimeter to about 0.20 of a millimeter, for the diameter of the non-absorbable synthetic fiber. When we utilize different materials that range may vary, however when we use the term ultra fine fiber we mean a fiber of diameter such that it does not cause scar tissue formation and would typically be less that 0.35 millimeter in diameter.

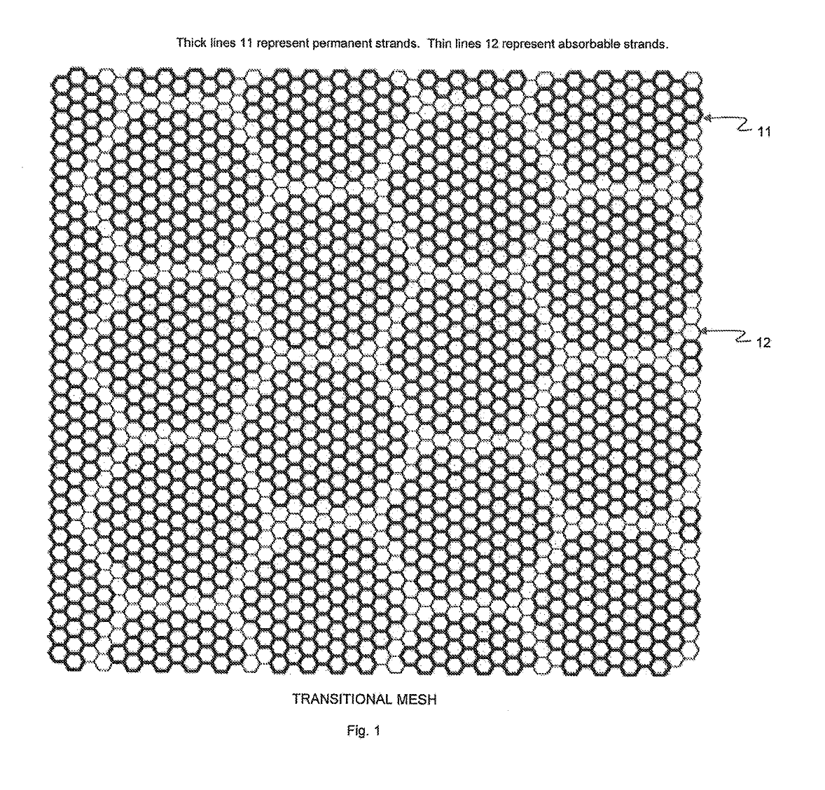

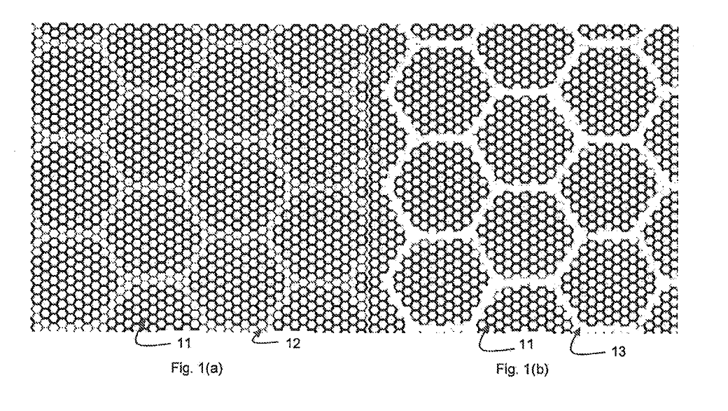

FIG. 1 shows a segmented fabric comprising sections which are permanent threads or strands, i.e. non-absorbable 11 and sections which are absorbable 12, such that the fabric transitions from a continuous sheet to an array of non-absorbable sections which are not connected to each other. We call this a transitional fabric mesh since the fabric changes its structural composition. The term “transitional” can also apply to a fiber or thread which utilizing this invention will also change its structural composition.

The absorbable material 11 generally would be, but is not required to be, of greater stiffness and strength to that of the non-absorbable fiber, allowing the fabric to provide support to the implant during the healing process. As the absorbable portion dissipates, the implant and tissue are provided structural support by the collagen structure formed about the non-absorbable fiber portions of the fabric 12. The areas between the non-absorbable segments, if such configuration is used, are natural body tissues that can move naturally and elastically. Since the absorbable fabric areas dissipate and provide disruptions to a continuous type of structure, and the fiber so small in diameter, there is virtually no scar tissue formed and capsular contracture does not become a problem. FIG. 1(a) shows the transitional mesh material when initially utilized adjacent to a view FIG. 1(b) of the transitional mesh material after absorption takes place. FIG. 1(b) shows the areas 13 where the absorbable portion of the fabric has dissolved. The words of “thread” and “fiber” are used interchangeably throughout and are to have the same meaning although a thread may be made of several fibers.

The transitional mesh fabric shown in FIG. 1 can be fabricated with the absorbable material placed or woven between the non-absorbable portions, or it can be constructed such that a fabric of absorbable materials is pressed or overlaid upon or encapsulate the non-absorbable components.

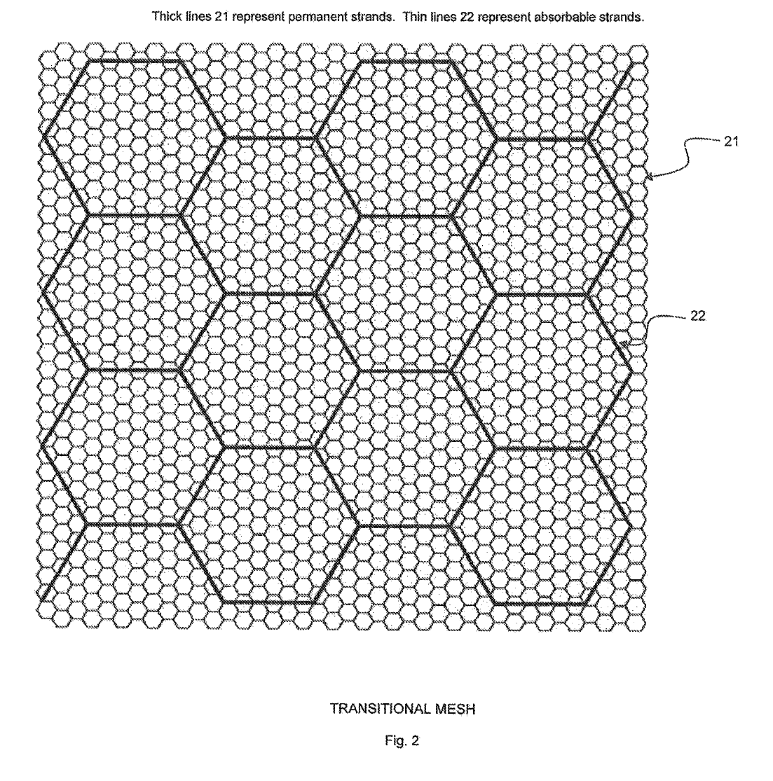

A detailed view of an alternate embodiment of a non-absorbable segment overlaid with absorbable material is show in FIG. 2, which shows a square section of another preferred embodiment where each sectional square is composed of non-absorbable material 21 which is threaded or woven into or superimposed upon a mesh of absorbable material 22.

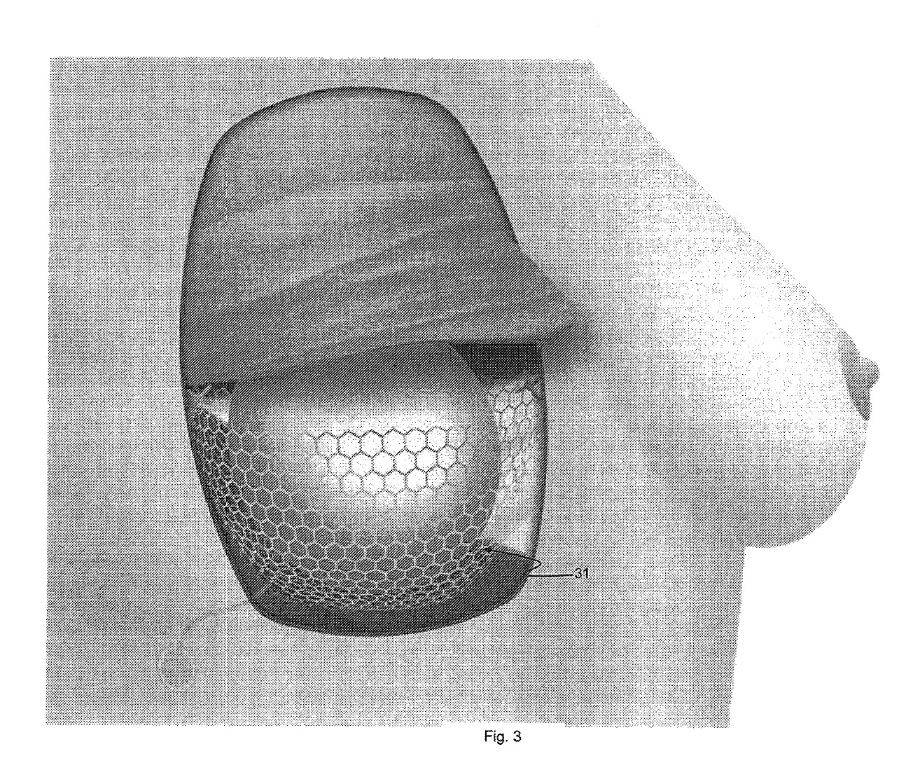

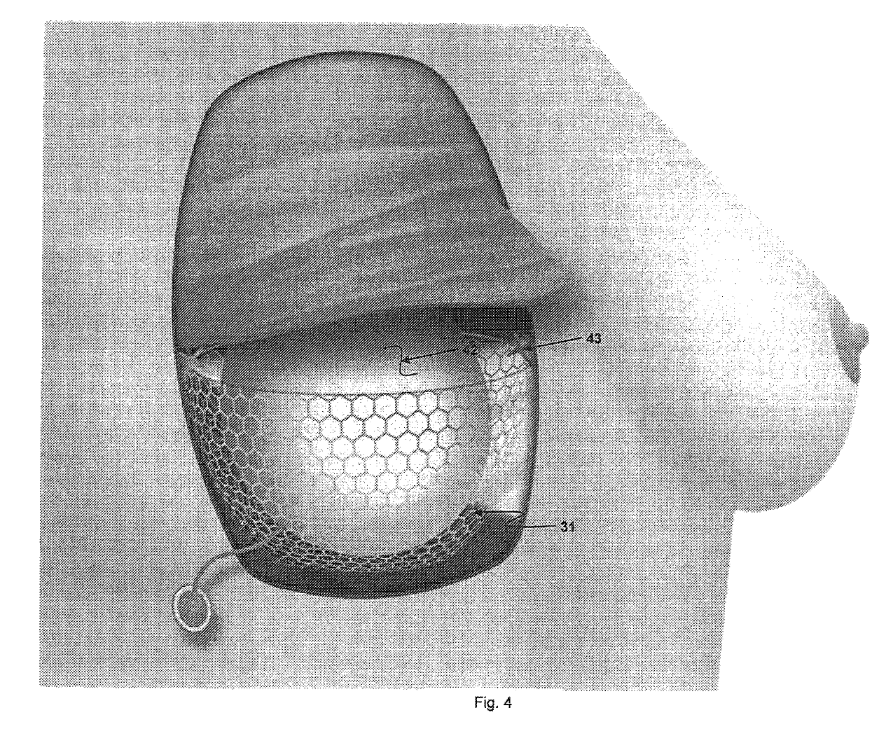

The transitional fabric can be utilized in typical breast implant procedures shown in FIGS. 3 and 4. In FIG. 3 we see the transitional mesh 31 formed in a cone shape to provide support to the implant. In FIG. 4 we see the transitional mesh 41 formed into a bag or pocket to contain the implant 42. The sutures 43 confine the movement of the mesh and thereby provide support to the implant during healing, with the non-absorbable components remaining to provide stimulus for forming the collagen structure to then provide continuing support. Absorbable hooks or transitional thread made up of alternate sections which are absorbable and sections which are non-absorbable can be utilized for sutures as the surgeon desires.

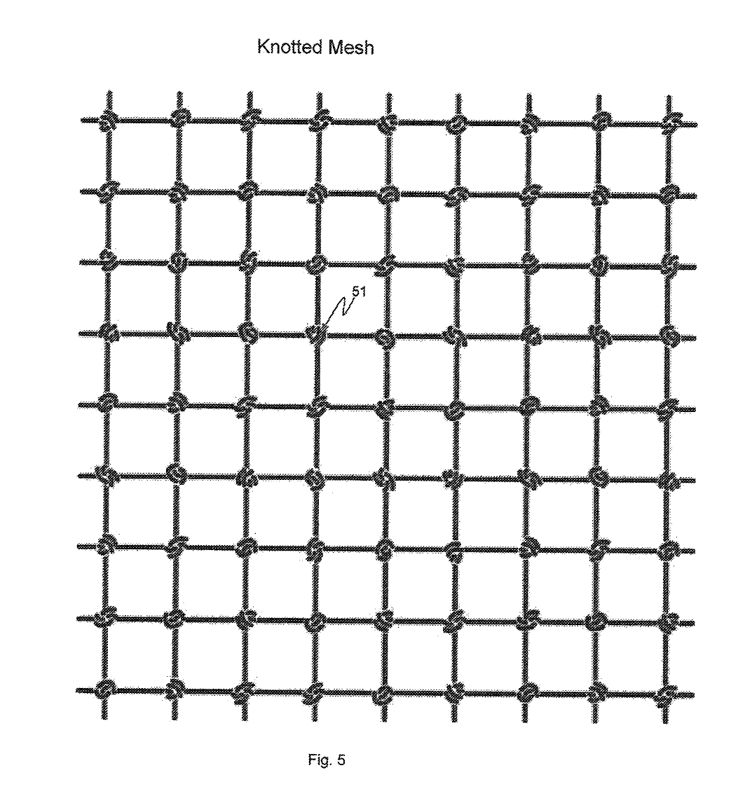

The mesh as shown in FIG. 5 is knotted, wherein each thread in the horizontal and vertical intersection 51 is knotted or fixed with the material of the thread having an inherent elasticity representing a form of yielding mesh. This configuration can be utilized when greater strength is needed. Alternatively, as seen in FIG. 6, where greater movement is desired, a non-knotted thread 61 and/or broken thread 62 can be utilized. This representative of a yielding mesh where yielding is accomplished by the lack of noted connections and/or breaks in the threads. As will be shown the ability to yield and thereby provide a degree of freedom of motion to provide a more natural appearance can also be accomplished using the appropriate weaving of fibers. Both these fabric type arrays promote the formation of a collagen structure in the body which provides flexible structure and support as well as natural movement.

FIG. 7a shows transitional mesh comprising of woven 71 threads housing or covered with an absorbable material 72 which provides temporary support and upon absorption will leave only the woven thread in a woven fabric formation FIG. 7(b). The woven threads provide freedom of movement once the absorbable components 72 are dissipated by virtue of, the loose weaving of the thread components, using an elastic type thread, by using an expandable knitting of the threads or by having the thread components be discontinuous 73 or a combination thereof. The remaining non-absorbable ultra fine fibers act as a scaffold for collagen growth which will provide strength for the tissue yet allow a high degree of freedom of motion.

The present invention, in a preferred embodiment FIG. 7(c), shows the absorbable and non-absorbable ultra fine fiber where the ultra fine fiber non-absorbable material is housed or covered with the absorbable material and the strands of non-absorbable fiber 71 remain after absorption FIG. 7(d). The elasticity, or ability to elongate, as well as strength is obtained by the formation of collagen forming over the ultra fine fibers. The ultra fine fibers being discontinuous and having breaks in its continuity allows greater movement. The non-absorbable ultra fine thread components 71 in FIG. 8(a) can also be wrapped or woven around the absorbable material 72 and in FIG. 8(b) we see the remaining non-absorbable loosely weaved thread 71 after absorption has occurred. The differing types of weaving or threading, size and type of thread, being monofilament or not, and patterns utilized gives one skilled in the art flexibility to select the type of material to meet the specific requirements needed.

The area between the non-absorbable segments can contain threads which have non-absorbable ultra fine fiber components if additional support is desired in the tissue between the non-absorbable segments.

This concept of transitional mesh can be applied to mesh of all types of patterns and sizes to allow its utilization for large areas where a greater portion of the area is composed of the non-absorbable material to smallest areas where the material is reduced to a transitional thread which is composed of a series of non-absorbable sections separated by absorbable sections. The thread when utilized results in discrete, non-absorbable sections remaining in the body to provide the basis for collagen formation to provide support. The shape shown of the non-absorbable mesh segments in the preferred embodiment in FIG. 1 and FIG. 2 is hexagonal, but that geometric shape is not required to obtain the benefits desired. Depending on the area, rectangular sections, square sections, random fibers and various other patterns or threads can also be employed to achieve the desired result of the invention.

It would be desirable but not required that the areas where non-absorbable fiber is utilized be staggered, or not on continuous straight lines or curves, as more natural motion can be obtained, and discontinuous non-absorbable fiber reduces formation of scar tissue.

The fabric fibers and materials in all of the embodiments may also be impregnated or coated with infection-fighting antibiotics or drugs to provide pain relief, to facilitate reduction of scar tissue, or to promote tissue in-growth.

Although the configurations in FIGS. 1 and 2 show the structure of the transitional mesh in two dimensions, that is not a required constraint. The mesh used for the absorbable and non-absorbable segments can have a varied height of surface which promotes tissue in-growth with the segment and helps impede scar tissue formation. Further, the segment or segments of non-absorbable fiber can be varied in size from small to large sections to accomplish the desired support needed.

Although use of the invention has been primarily shown for breast implant utilization, the transitional mesh fabric can be used for other surgical repairs or utilized in areas where additional strength is needed but natural motion and minimal scar tissue formation is desired.

The types of mesh and fiber that can be utilized can vary depending upon the needs and desires of the surgeon. The material utilized for the fabrics in the preferred embodiments are composed of natural and synthetic materials, parts bio-absorbable and parts non-bio-absorbable. When used in this patent, the term “absorbable” incorporates “bio-absorbable” and “bio-degradable” and means the composition of the material is broken down or assimilated by the human body and there is no stiffness nor significant tensile strength remaining in the material. The final disposition of the material may or may not result in it being ultimately flushed from the body's tissue. The transitional mesh material may also be composed of threads which themselves are partially absorbable and lose tensile strength. Other fabric materials available include those that are totally biological in nature, e.g. collagen, skin tissue, or a combination of biological and synthetic materials. Variations in type of mesh and material also exist within the mesh structure where some thread materials are knotted, some without knots and the strands are threaded, braided or woven.

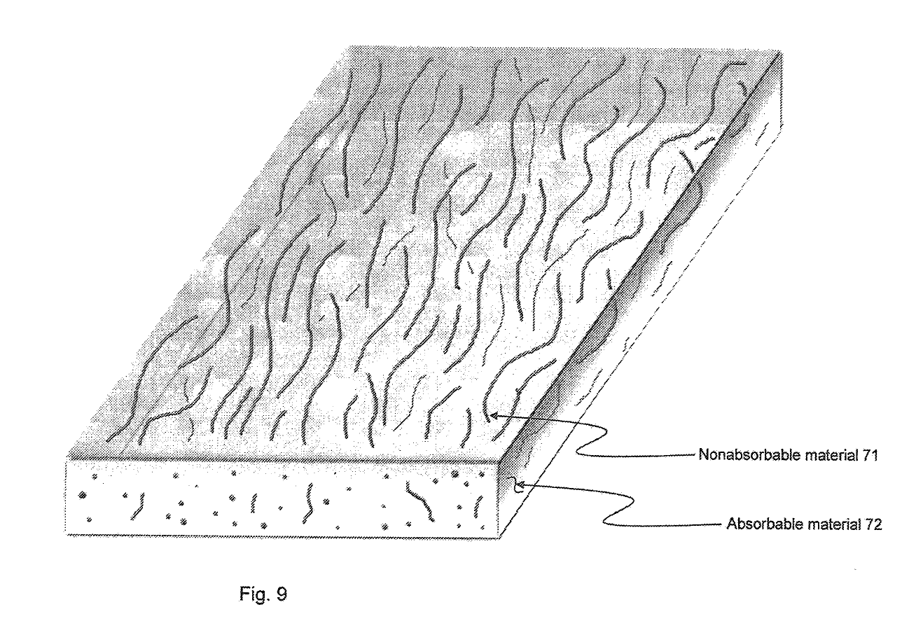

Another preferred embodiment FIG. 9 would have the absorbable material in a soft sponge-like consistency 72 being formed as a sheet which has intermixed within the absorbable material 72 threads or fibers of non-absorbable material 71. The absorbable material dissipates and collagen forms about the remaining non-absorbable ultra fine fibers. Although in FIG. 9 the non-absorbable fibers are shown to run in the direction of one axis, they can be running in any direction or in a random array depending upon the effect you wish to obtain. Permanent synthetic materials can be, but are not limited to, nylon, prolene, polyester, and polytetra flourethylene (PTFE). Absorbable materials may be, but are not limited to cat gut, polyglysolic acid, trimethylene carbonate, and silk. The types of materials used for both non-absorbable material and absorbable material are constantly changing and would be known to one skilled in the art.

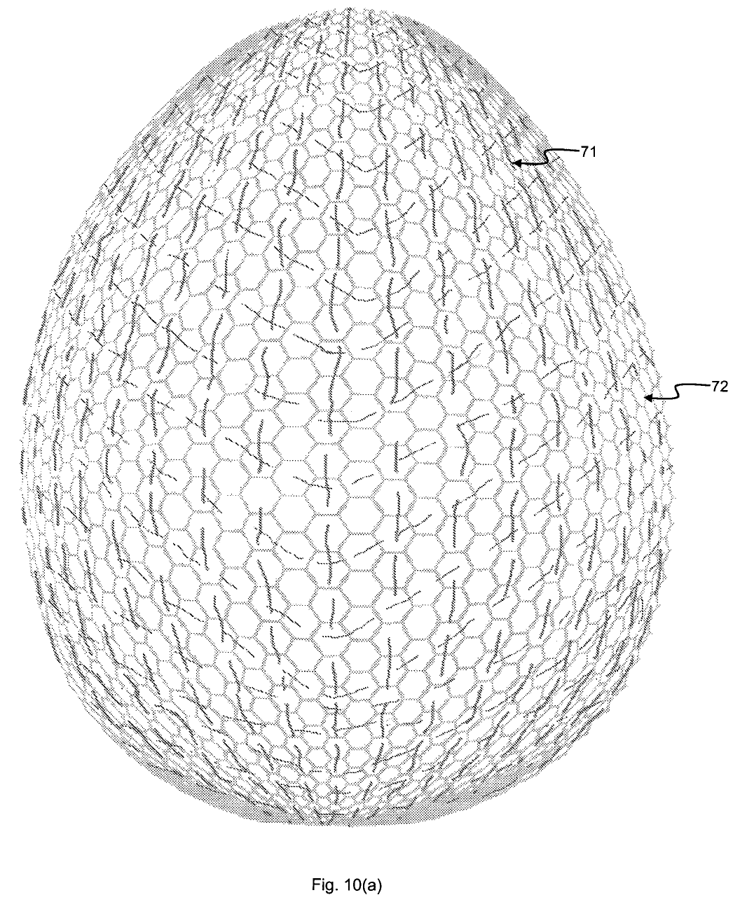

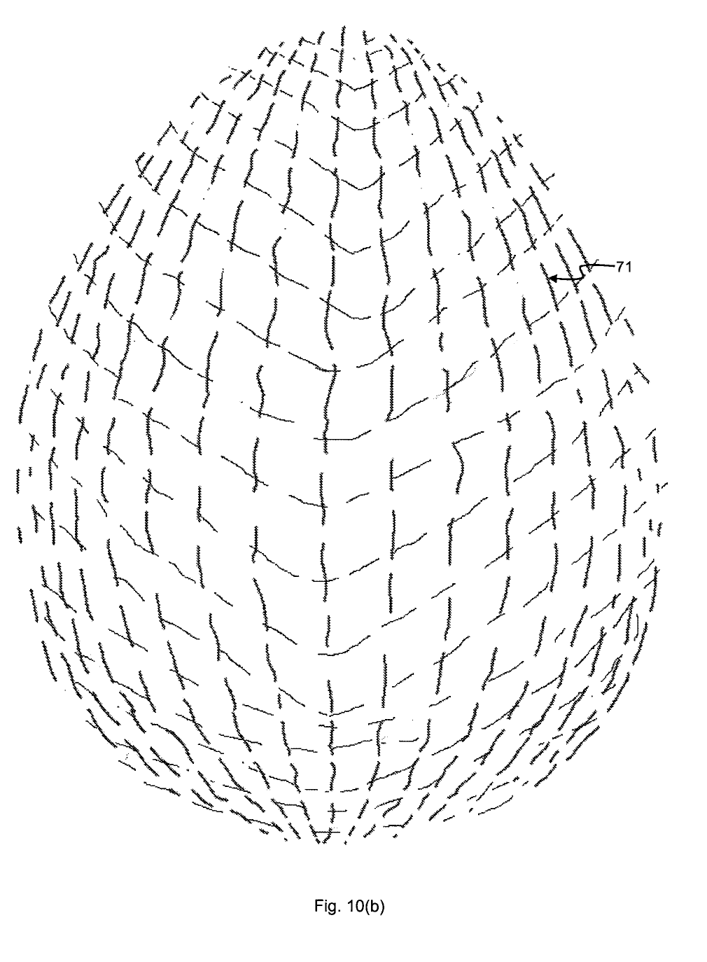

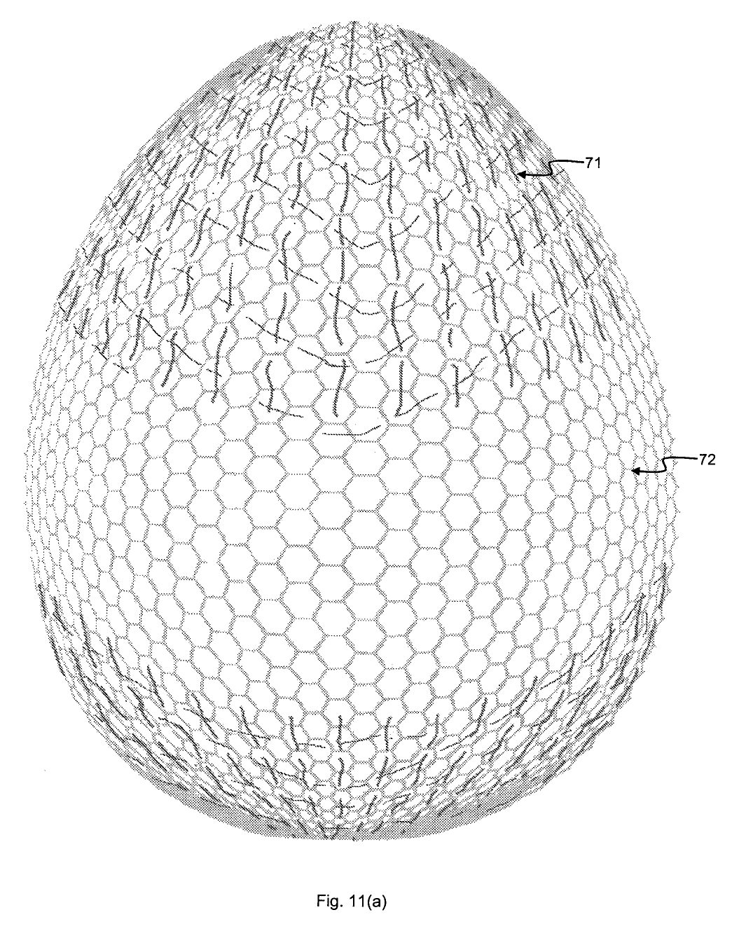

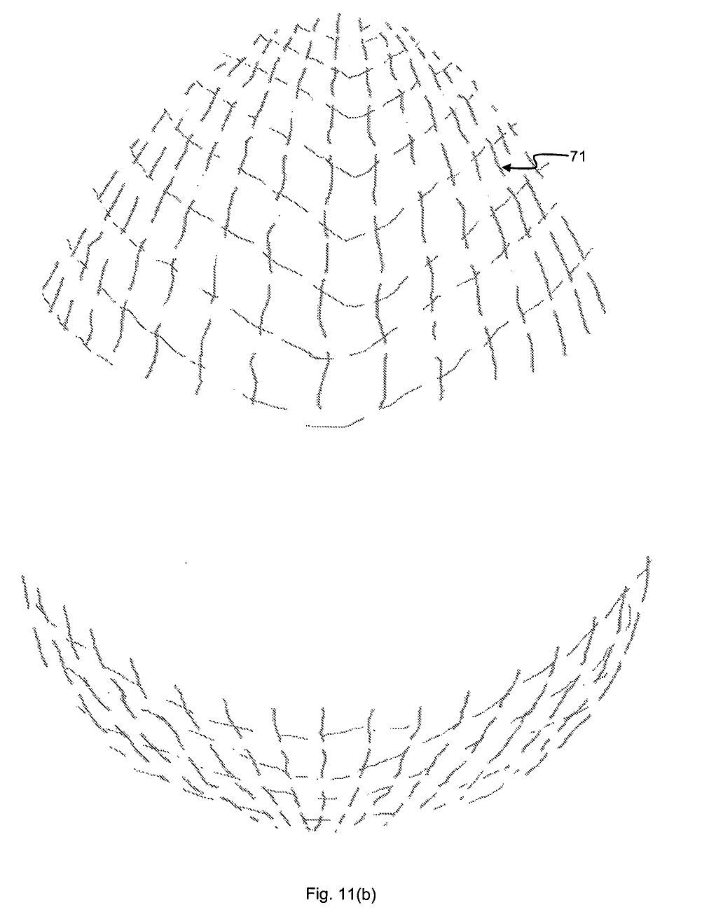

An alternate construction and usage is shown in FIG. 10(a) and FIG. 11(a). In FIG. 10(a) the absorbable material 72 is in a mesh configuration covering the entire area or device with non-absorbable thread 71 overlaid or interwoven throughout the mesh material in a non-continuous pattern. The pattern can also be a random array. Upon utilization the absorbable material 72 will dissolve leaving the non-absorbable material and collagen to provide support. FIG. 10(b). The gradual transfer of tension load from the absorbable material to the collagen structure causes the collagen structure to strengthen to perform the needed support and not be absorbed into the body. The amount and configuration of the non-absorbable material can be selected to obtain the desired collagen support. Another variation is shown in FIG. 11(a) where non-absorbable 71 in conjunction with absorbable material is only in predetermined areas and other areas are only absorbable material 72. As shown in FIG. 11(b) the non-absorbable material 71 will ultimately provide the collagen growth to support the specific areas desired. FIG. 11(b). When the ultra fine fiber is used the support initially will be provided by the absorbable material. Variations of this configuration can provide the desired support in particular applications.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

1. A biocompatible fabric material for implanting into the body to provide support comprising: a plurality of non-absorbable ultra fine fibers, embedded in an absorbable material, wherein the sections are placed over the desired area.

2. The non-absorbable ultra fine fibers of claim 1, wherein the non-absorbable ultra fine fibers are comprised of fibers with a diameter less than 0.30 of a millimeter.

3. The non-absorbable ultra fine fibers of claim 1, wherein the non-absorbable ultra fine fibers are comprised of fibers with a diameter less than 0.35 of a millimeter.

4. The non-absorbable ultra fine fibers of claim 1, wherein the non-absorbable ultra fine fibers are coated with absorbable material.

5. The non-absorbable ultra fine fibers of claim 1, wherein the non-absorbable ultra fine fibers are comprised of fibers with a diameter between about 0.01 of a millimeter and about 0.10 of a millimeter.

6. The material of claim 1, wherein the non-absorbable threads are knotted.

7. The material of claim 1, wherein the non-absorbable sections are coated with an antibiotic material.

8. The material of claim 1, wherein the non-absorbable sections are coated with material to reduce scar tissue formation.

9. The material of claim 1, wherein the non-absorbable sections are coated with material to promote tissue growth.

10. The material of claim 1, wherein the non-absorbable mesh sections are coated with a medicinal material.

11. A biocompatible fabric of material for implanting into the body to provide support comprising: a plurality of non-absorbable synthetic fibers, wherein the non-absorbable synthetic fibers are placed in an array; and portions of space between adjacent non-absorbable synthetic fibers contain absorbable material.

12. The fabric of claim 11, wherein the non-absorbable synthetic fibers are surrounded by absorbable material at perimeter edges.

13. The fabric of claim 11, wherein the non-absorbable synthetic fibers have a diameter of less than 0.35 of a millimeter.

14. The fabric of claim 11, wherein the wherein the non-absorbable synthetic fibers have a diameter of about 0.01 of a millimeter to about 0.10 of a millimeter.

15. The fabric of claim 11, wherein the wherein the non-absorbable synthetic fibers have a diameter of less than 0.20 of a millimeter.

16. The fabric of claim 11, wherein the non-absorbable synthetic fibers are coated with material to reduce scar tissue formation.

17. The fabric of claim 11, wherein the woven fabric is coated with material to promote tissue growth.

18. The fabric of claim 11, wherein the woven fabric is infused with a medicinal material.

19. A biocompatible sheet material for implanting into a body to provide support comprising:
an absorbable material in the form of a sheet; and a plurality of non-absorbable synthetic fibers, wherein the non-absorbable synthetic fibers are of a diameter between about 0.01 of a millimeter and about 0.20 of a millimeter distributed within the absorbable material.

20. A biocompatible sheet material for implanting into a body to provide support comprising: an absorbable material in the form of a sheet; and a plurality of non-absorbable synthetic fibers, wherein the non-absorbable synthetic fibers are of a diameter less than 0.35 of a millimeter distributed within the absorbable material,

21. The material of claim 19, wherein the consistency of the absorbable sheet material is sponge-like.
